(19) United States
(12) Patent Application Publication
(10) Pub. No.: US 2006/0116584 A1
(43) Pub. Date: Jun. 1, 2006

(54) Miniaturized ultrasonic transducer

(75) Inventors: Wojtek Sudol, Andover, MA (US); Ed Gurrie, North Andover, MA (US); Bernard Savord, Andover, MA (US); Martha Wilson, Andover, MA (US); Showna Chang, Somerville, MA (US)

Correspondence Address: PHILIPS INTELLECTUAL PROPERTY & STANDARDS, P.O. BOX 3001, BRIARCLIFF MANOR, NY, 10510, US

(73) Assignee: KONINKLIJKE PHILIPS ELECTRONIC N.V., Eindhoven (NL)

(21) Appl. No.: 10/537,891

(22) PCT Filed: Nov. 24, 2003
(86) PCT No.: PCT/IB03/05418

§ 371 Date: Jun. 7, 2005

Related U.S. Application Data
(60) Provisional application No. 60/432,536, filed on Dec. 11, 2002.

(51) Int. Cl. A61B 8/14

(52) U.S. Cl. 600/459

(57) ABSTRACT

Ultrasonic transducers having a reduced size in comparison with prior art ultrasonic transducers and including a thermally-conductive body, a flexible circuit bent at least partially around the body, an acoustic assembly arranged on the flexible circuit and electronic components for controlling the acoustic assembly to transmit and receive ultrasonic waves. Signal transmission lines, such as coax wires, are coupled to the flexible circuit such that the electronic components, the acoustic assembly and the signal transmission lines are connected in a circuit defined in part by the flexible circuit. By bending the flexible circuit with the acoustic assembly, and optionally the electronic components, arranged thereon about the body, they are positioned in a vertical configuration which allows for a compact transducer which has a small, even miniature size in comparison to prior art ultrasonic transducers.

### FIELD OF THE INVENTION

The present invention relates generally to ultrasonic transducers having a sufficiently small size to enable their use in small medical instruments, in particular, transesophageal examination devices, laproscopic examination devices and intra-cardiac examination devices, and more particularly to such ultrasonic transducers having acoustic elements mounted over an integrated circuit.

The present invention also relates to methods for manufacturing ultrasonic transducers having a size small enough to enable their use in medical instruments, in particular, transesophageal examination devices, laproscopic examination devices and intra-cardiac examination devices.

### BACKGROUND INFORMATION

A typical ultrasonic transducer used in a medical instrument for imaging portions of the body to generate a three-dimensional image has a complicated interconnection of the various components of the transducer. As a result, it has proven to be costly to build such transducers. Moreover, it is a drawback of such transducers that in view of the complicated interconnection of components, they require a relatively large amount of space and therefore cannot be used in applications where a very small or miniature ultrasonic transducer is needed, such as for examining the esophagus and heart and other relatively small parts of the body.

Thus, while such transducers can be used as transthorasic transducers, they cannot be used as transesophageal transducers, laproscopic transducers and intra-cardiac transducers because they are too large.

### SUMMARY OF THE INVENTION

It is an object of the present invention to provide a new and improved ultrasonic transducer which has a very small, miniature size.

It is another object of the present invention to provide a new and improved ultrasonic transducer having a sufficiently small size to enable its use in small medical instruments, in particular, transesophageal examination devices, laproscopic examination devices and intra-cardiac examination devices.

It is yet another object of the present invention to provide a new and improved ultrasonic transducer which includes a flexible circuit thereby enabling the size of the transducer to be reduced in comparison with prior art ultrasonic transducers.

It is still another object of the present invention to provide a new and improved method for manufacturing ultrasonic transducers having a size small enough to enable their use in small medical instruments, in particular, transesophageal examination devices, laproscopic examination devices and intra-cardiac examination devices.

In order to achieve these objects and others, an ultrasonic transducer in accordance with the invention comprises a thermally-conductive body, a flexible circuit bent at least partially around the body, an acoustic assembly connected to the flexible circuit and electronic components for controlling the acoustic assembly to transmit and receive ultrasonic waves. Signal transmission lines or conduits, such as coax wires, flat ribbon cables or long flexible circuits, are coupled to the flexible circuit such that the electronic components, the acoustic assembly and the signal transmission lines are connected in a circuit defined in part by the flexible circuit. The electronic components and acoustic assembly are optionally arranged on the flexible circuit. By bending the flexible circuit with the acoustic assembly and the electronic components arranged thereon about the body, they are positioned in a vertical configuration which allows for a compact transducer which has a small, even miniature size in comparison to prior art ultrasonic transducers.

More particularly, the flexible circuit is bent around the body such that one part having the acoustic assembly arranged thereon is on a first side of the body and a second part having the electronic components arranged thereon is on a second, opposite side. A 180° bend around a leg portion of the body separates the two parts of the flexible circuit. Additional bends are provided to enable terminal end portions of the flexible circuit to be vertically spaced from the body arrangement, with the signal transmission lines being coupled to the terminal end portions, possibly by means of additional flexible circuits. Preferably, the electronic components are positioned in a cavity defined by the body. The part of the flexible circuit to which the electronic components are mounted may be positionable in the cavity as well.

In one embodiment, the acoustic assembly includes acoustic elements and an integrated circuit electrically coupled to the acoustic elements. The integrated circuit is also electrically coupled to the flexible circuit. Specifically, the flexible circuit and the integrated circuit each have connection sites or connector pads with wire-bonds being provided to connect the connection sites of the integrated circuit and the flexible circuit.

Another embodiment of an ultrasonic transducer in accordance with the invention includes a housing, acoustic elements arranged in the housing and an integrated circuit arranged in the housing adjacent the acoustic elements and connected to the acoustic elements. The integrated circuit is connected to electrical transmission lines. Connection sites for the connections to the integrated circuit are arranged on a common surface thereof. More specifically, the integrated circuit may be connected to the acoustic elements and the signal transmission lines using metal bumps, solder bumps, polymer bumps, thin-line bonding, z-axis conductive elastomeric connectors, z-axis conductive adhesive, z-axis conductive film and/or reflow solder. In addition, the integrated circuit may be coupled to an intermediate interconnection substrate, such as an at least partially flexible circuit, using wire-bonds, direct wire attachments and/or tab bonding of leads. The interconnection substrate may also be a thin film circuit or ceramic circuit and/or use laminate circuit technology. Still another embodiment of an ultrasonic transducer in accordance with the invention includes a flexible circuit having connection sites, an acoustic assembly mounted on the flexible circuit and an integrated circuit having connection sites and acoustic elements electrically coupled to the integrated circuit, and electronic components for controlling the acoustic assembly to cause the acoustic assembly to transmit and receive ultrasonic waves. Wire-bonds are formed to connect the connection sites of the integrated circuit and the connection sites of the flexible circuit. The acoustic assembly and electronic components are thus connected in a circuit defined in part by the flexible circuit. The wire-bonds may be formed along only a portion of the periphery of the integrated circuit. In one embodiment, two rows of wire-bonds are formed along each of a pair of opposed edges of the integrated circuit.

In accordance with another embodiment of the invention, a method for manufacturing miniature ultrasonic transducers includes the steps of arranging an acoustic assembly on a flexible circuit, e.g., when the flexible circuit is flat, coupling electronic components for controlling the acoustic assembly to the acoustic assembly circuit, coupling signal transmission lines to the flexible circuit such that the electronic components, the acoustic assembly and the signal transmission lines are connected in a circuit defined in part by the flexible circuit and bending the flexible circuit at least partially around a thermally-conductive body to form at least one 180° bend about the body. When the electronic components are also mounted on the flexible circuit, after the bending of the flexible circuit about the body, the acoustic assembly will be vertically spaced from the electronic components. In this manner, the acoustic assembly and electronic components are in a vertical arrangement one substantially above the other so that a compact transducer is provided which has a sufficiently small size to enable its use in transesophageal examination devices, laproscopic examination devices and intra-cardiac examination devices.

These and other objects, features and advantages of the present invention will be explained below with reference to the following drawings.

### BRIEF DESCRIPTION OF THE DRAWINGS

FIG. 1 is a cross-sectional view of a transducer in accordance with the invention shown in the outline of a tip of a transesophageal examination probe;

FIG. 2 is an illustration of an acoustic assembly in which acoustic elements are mounted over an integrated circuit;

FIG. 3 is an enlarged view of a first embodiment of the section designated 3 in FIG. 2.

FIG. 4 is an enlarged view of a second embodiment of the section designated 3 in FIG. 2;

FIG. 5 is a top view of the transducer in accordance with the embodiment of the invention shown in FIG. 1;

FIG. 6 is a cross-sectional view of another embodiment of a transducer in accordance with the invention shown in the outline of a tip of a transesophageal examination probe;

FIG. 7 is a cross-sectional view of another embodiment of a transducer in accordance with the invention shown in the outline of a tip of a transesophageal examination probe;

FIG. 8 is a sectional view taken along line 8-8 of FIG. 7; and

FIG. 9 is a cross-sectional view of another embodiment of a transducer in accordance with the invention shown in the outline of a tip of a transesophageal examination probe.

### DESCRIPTION OF THE INVENTION

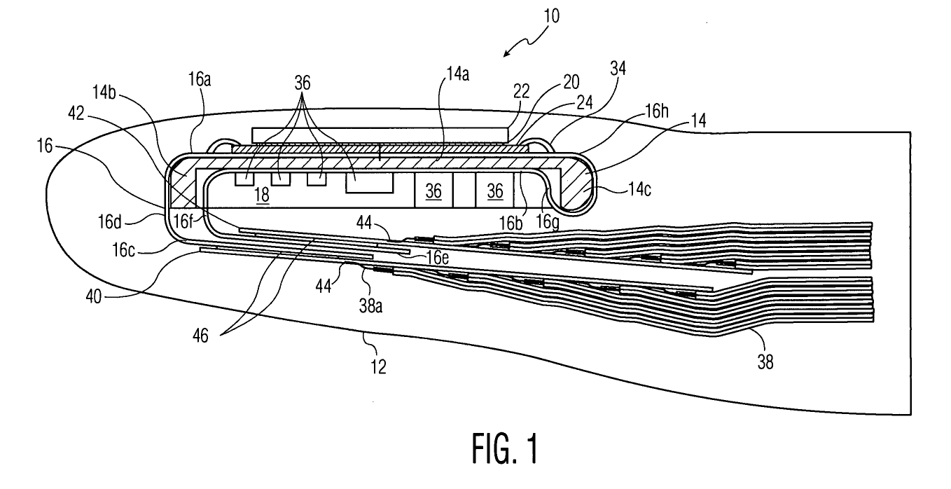

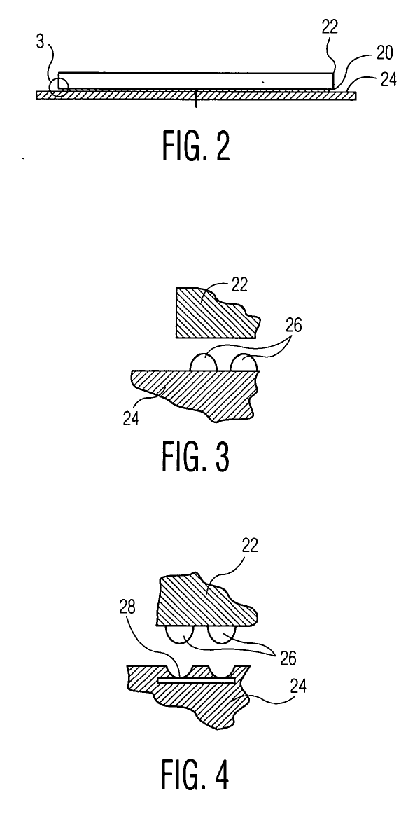

Referring to the accompanying drawings wherein like reference numerals refer to the same or similar elements, FIG. 1 shows a first embodiment of an ultrasonic transducer in accordance with the invention which is generally designated as 10. The ultrasonic transducer is small enough to fit within the tip of a standard-size transesophageal examination probe, represented by the line 12, or another similarly-sized or smaller probe housing. Previously, miniaturization of an ultrasonic transducer to fit within the tip of such a device would not be possible.

To achieve this miniaturization, the transducer 10 includes a thermally-conductive body 14 and a flexible circuit 16 which is bent around the body 14. By providing the flexible circuit 16 and coupling the components necessary for operation of the transducer 10 to the flexible circuit 16, the flexible circuit 16 can be bent into a desired shape to enable it to fit within the tip 12 of the examination device. The flexible circuit 16 is a laminate including electrically-conductive paths and connection sites enabling electrical connection to electrical components. As described below, it serves an intermediate interconnection substrate for connecting an integrated circuit to signal transmission lines. The flexible circuit 16 is bent around the body 14 which has a substantially U-shaped cross-section at the portion around which the flexible circuit 16 is bent and thereby defines a cavity 18. The body 14 has a central support portion 14a and leg portions 14b, 14c, one at each end of the support portion 14a, with the flexible circuit 16 being supported by the support portion 14a and bent over the leg portions 14b, 14c.

The flexible circuit 16 is not required to be flexible over its entire length to achieve the objects of the invention, although it is a possibility. Rather, it suffices that those portions of the flexible circuit 16 which are bent, e.g., those portions bent over the leg portions 14b, 14c, are flexible. Other portions of the flexible circuit 16 which are not bent, such as those planar portions which support components of the transducer 10 described below, may be rigid. Thus, the flexible circuit 16 may be formed from a combination of one or more flexible circuit boards and one or more rigid circuit boards such as PCBs (printed circuit boards) or ceramic circuit boards.

As shown in FIG. 1, the cavity 18 is formed on the underside of the body 14. The flexible circuit 16 has a first planar portion 16a above the body 14, a second planar portion 16b situated in the cavity 18, a terminal end 16c separated from the first planar portion 16a by a one-hundred-eighty degree (180°) bend 16d and a second terminal end 16e separated from the second planar portion 16b by a one-hundred-eighty degree (180°) bend 16f. In the embodiment shown in FIG. 1, the terminal ends 16c and 16e are substantially planar and situated at least partially opposite one another below the body 14. The flexible circuit 16 also includes a curved portion 16g adjacent the portion 16b in the cavity 18 and a one-hundred-eighty degree (180°) bend 16h between the portion 16a above the body 14 from the curved portion 16g.

The one-hundred-eighty degree (180°) bends 16d, 16f and 16h may include a pair of ninety degree (90°) bends separated by a straight portion as shown in FIG. 1 or be entirely arcuate. The form of the bends would depend on the shape of the body 14. In general, the flexible circuit 16 is bent so as to provide one portion above the body 14 and one portion below the body 14.

An acoustic assembly 20 is mounted to an upper surface of the first planar portion 16a of the flexible circuit 16. Although the acoustic assembly 20 may be any type of known acoustic assembly for transmitting and receiving ultrasonic waves, in a preferred embodiment, the acoustic assembly 20 includes a stack of acoustic elements 22 connected to connector pads or sites on the upper surface of an integrated circuit 24 using a flip-chip interconnection technique, the specific details of which will be apparent to one of ordinary skill in the art. The number of interconnections between the acoustic elements 22 and the integrated circuit 24 may vary depending on the number of acoustic elements 22 and the size and shape of the acoustic elements 22 and integrated circuit 24 and may even be as high as in the order of about 3000. The acoustic elements 22 may be arranged in a linear array, i.e., a line of acoustic elements to provide a one-dimensional transducer, or in a multi-dimensional array, e.g., a two-dimensional matrix of acoustic elements to provide a two-dimensional transducer. The acoustic assembly 20 may be planar or curved.

Other methods for connecting the acoustic elements 22 to the integrated circuit 24 include the use of metal, solder or polymer bumps 26 (as shown in FIGS. 3 and 4), thin-line bonding, z-axis conductive elastomeric connectors, z-axis conductive adhesive, z-axis conductive film and reflow solder. In FIG. 3, the bumps 26 are formed on the integrated circuit 24 whereas in FIG. 4, the bumps 26 are formed on the acoustic elements 22 and openings 28 are formed in the upper surface of the integrated circuit 24 to enable contact with a conductive layer in the integrated circuit 24. Reverse flip-chip interconnection techniques can also be used.

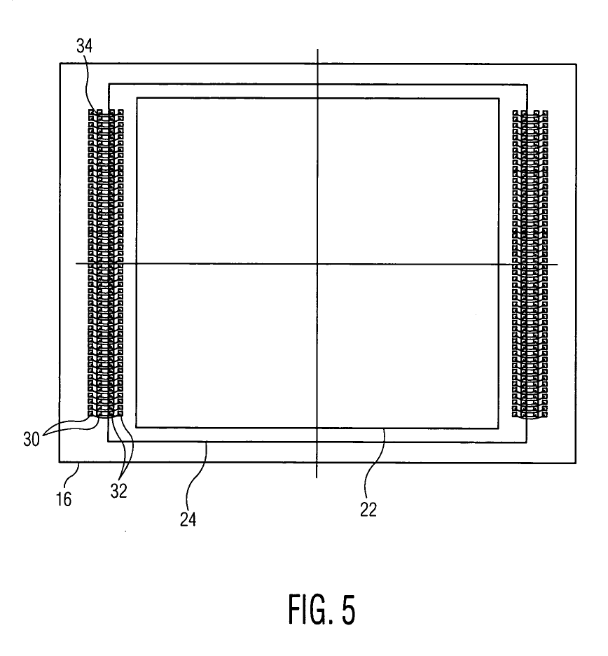

As shown in FIG. 5, the integrated circuit 24 is connected to the flexible circuit 16 by wire-bonding, i.e., connection sites or connector pads 30 on the flexible circuit 16 are connected to connection sites or connector pads 32 on the upper surface of the integrated circuit 24 by short wires 34 (also referred to as wire-bonds). Thus, the electrical connections, i.e., the connector pads or sites, for the acoustic elements 22 and for the flexible circuit 16 are both arranged on the upper surface of the integrated circuit 24. Nevertheless, the connections may be arranged on different surfaces in other embodiments. The wire-bonding between the flexible circuit 16 and the integrated circuit 24 can be provided all around the periphery of the integrated circuit 24 or as shown in FIG. 5, only along one or more discrete portions of the periphery. More specifically, as shown in FIG. 5, on a pair of opposite sides of the integrated circuit 24, there are two rows of wire-bonds (also referred to as a double-row). By having multiple rows of wire-bonds 34 on only a pair of opposite sides of an integrated circuit 24, a more ergonomic design of the transducer 10 is provided, i.e., a narrower transducer.

Instead of wire-bonds, a direct wire attachment or tab bonding of leads can be provided between the connector pads 30 and the connector pads 32.

Preferably, the integrated circuit 24 is situated as close as possible to the body 14 to provide a short heat path to the body 14. A short heat path between the integrated circuit 24 and the body 14 enables heat generated by the integrated circuit 24 to be transferred to the body 14 and dissipated thereby. The body 14 thus serves as a heat sink and accordingly is made of materials which have good thermal conductivity such as copper, aluminum, brass, graphite and mixtures thereof, or other thermally conductive materials.

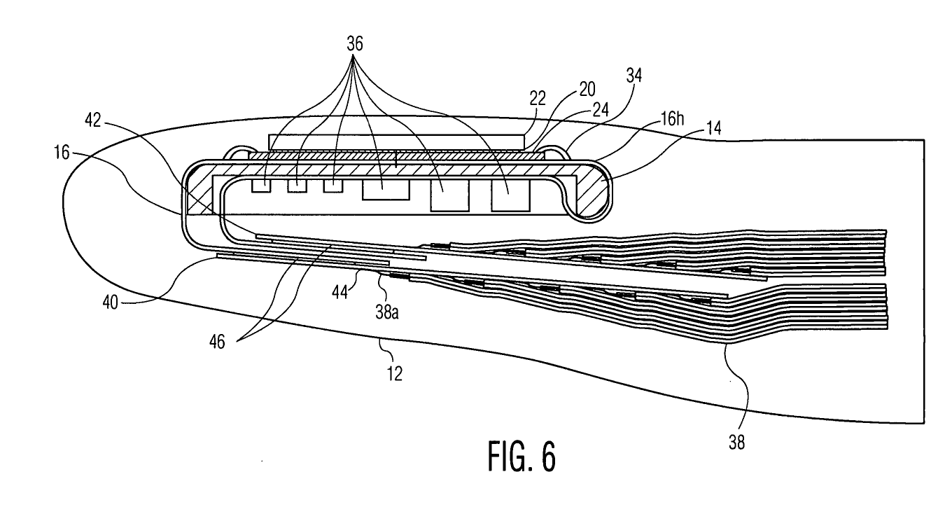

In one embodiment shown in FIG. 6, the integrated circuit 24 is in direct contact with the body 14, thereby providing the shortest possible heat path. This is made possible by forming the flexible circuit 16 around the integrated circuit 24.

Electronic operational components 36 required for operation and control of the transducer 10 are mounted to the second planar portion 16b of the flexible circuit 16 in any manner known in the art, e.g., by surface-mounting, such that the components 36 are located in the cavity 18. Typically, there may be ten or more such components. The components 36 are thus situated in the cavity 18 and do not project beyond the lower surface of the body 14. It should be noted that in view of the bending of the flexible circuit 16 about the body 14, the acoustic assembly 20 and components 36 are mounted on the same side of the flexible circuit 16 during the manufacture of the transducer 10 (described below).

The reduction in the vertical size of the transducer 10 is obtained when the flexible circuit 16 is bent. In one embodiment, the flexible circuit 16 may be bent until the vertical size of an assembly of the flexible circuit 16 (bent around the body 14), the acoustic elements 22 and the integrated circuit 24 is less than seventy-five percent, or even less than fifty percent, of the horizontal length of the integrated circuit 24.

To connect the flexible circuit 16 to a plurality of coax wires 38 leading from the examination device to associated equipment, such as a monitor and recording device, a pair of additional flexible circuits 40,42 is used, each having appropriate connections for coax wires 38 such as connection sites or connector pads 44. The number of coax wires 38 may vary depending on the application of the transducer 10 but may be as high as 160 or even as high as 200. Each flexible circuit 40,42 is connected to a portion of the coax wires 38 by bonding exposed, conductive portions 38a of the coax wires 38 to the connection sites of the flexible circuits 40,42, e.g., using a known bonding process. The flexible circuits 40,42 may be entirely flexible or have a flexible portion and a rigid portion, and might even be entirely rigid.

Connection of the coax wires 38 to the flexible circuits 40,42 may be performed separate from the manufacture of the flexible circuit 16 with the acoustic assembly 20 and optional electronic components 36. This provides a significant advantage in view of the number of coax wires 38 because it enables separate manufacture of the flexible circuit 16 and associated componentry and of the connection mechanism for connecting the flexible circuit 16 to the external devices (the coax wires 38 and flexible circuits 40,42).

The flexible circuits 40,42 are connected to the flexible circuit 16 using an electrical interconnection such as z-axis conductive film or conductive adhesive 46. In this manner, an electrical connection between the flexible circuit 16 and the coax wires 38 is provided via the flexible circuits 40,42 and the adhesive 46. Instead of z-axis conductive film or adhesive, it is possible to use a z-axis conductive elastomeric connector or reflow solder.

Instead of mounting the electronic components 36 to the flexible circuit 16, electronic components or electronics for controlling the acoustic assembly 20 may be mounted on the flexible circuits 40,42 or at the end of the coax wires 38 distanced from the transducer 10. The electronic components could also be integrated into the integrated circuit 24.

To manufacture the transducer 10, the body 14 is formed and the flexible circuit 16 is formed and cut to the necessary size to enable it to be bent around the body 14. The acoustic assembly 20 and the electronic components 36 are mounted to the same side of the flexible circuit 16 in connection with or after the formation of the flexible circuit 16. To enable mounting of the acoustic assembly 20 to the flexible circuit 16, adhesive is applied to the underside of the integrated circuit 24. The mounting locations of the acoustic assembly 20 and electronic components 36 are selected to position the acoustic assembly 20 above the cavity 18 and the electronic components 36 in the cavity 18 as shown in FIG. 1. The connection sites 32 of the acoustic assembly 20 are then connected to the connection sites 30 of the flexible circuit 16 by wire bonds 34. The acoustic assembly 20 may be pre-formed by mounting the stack of acoustic elements 22 on the integrated circuit 24 and connecting them using a flip-chip interconnection technique.

Flexible circuits 40,42 are formed with the required connection sites for electrical connection with the flexible circuit 16 and the coax wires 38 and then attached to the coax wires 38, e.g., by soldering. The flexible circuits 40,42 are also attached to the terminal ends 16c, 1 6e of the flexible circuit 16 using z-axis conductive film or conductive adhesive 46. The flexible circuits 40,42 may be attached to the coax wires 38 first and then to the flexible circuit 16 or vice versa.

Once the acoustic assembly 20, electronic components 36 and flexible circuits 40,42 (preferably with the coax wires 38 attached thereto) are attached to the flexible circuit 16, adhesive is applied to the portions of the flexible circuit 16 which will come into contact with the body 14 (and/or applied to portions of the body 14 against which the flexible circuit 16 will rest) and then the flexible circuit 16 is bent around the body 14 such that the planar portion 16a of the flexible circuit having the acoustic assembly 20 mounted thereon is situated on the support portion 14a of the body 14, the planar portion 16b having the electronic components 36 mounted thereon is situated in the cavity 18 in the body 14, and the terminal portions 16c and 16e having the flexible circuits 40,42 attached thereto are situated underneath the body 14. Further, bending of the flexible circuit 16 over the body 14 is performed such that the bend 16d of the flexible circuit 16 is situated partially over the leg portion 14b of the body 14, the bend 16f is situated partially inside the cavity 18 of the body 14, the arcuate portion 16g is situated in the cavity 18 and the bend 16h is situated over the leg portion 14c of the body 14. The acoustic assembly 20, the electronic components 36 and the attachment mechanism for attaching the flexible circuit 16 to the coax wires 38 are thus all positioned in a vertical arrangement, vertically spaced from one another, thereby reducing the horizontal size of the transducer. In fact, it can be seen from FIG. 5 that the size of the transducer 10 is not much larger than the size of the integrated circuit 24. A compact transducer is thus provided which can fit in the tip of a transesophageal examination device (line 12 as shown in FIG. 1).

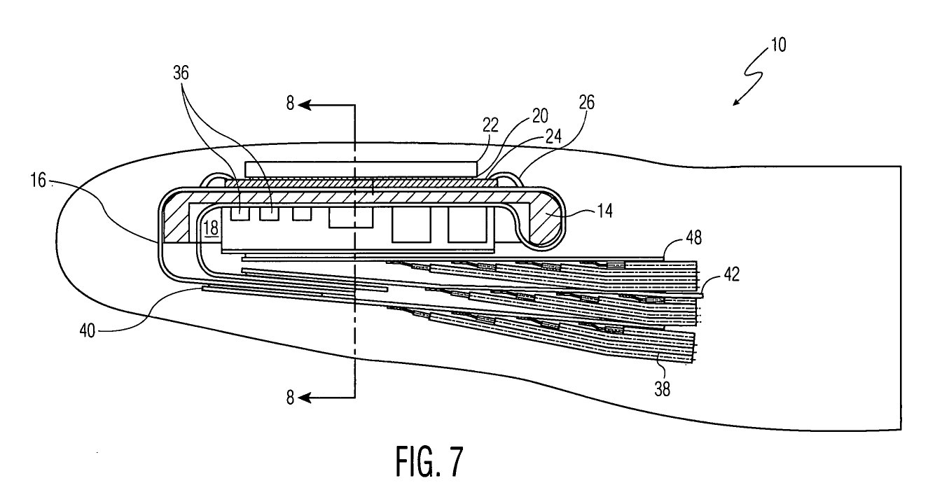

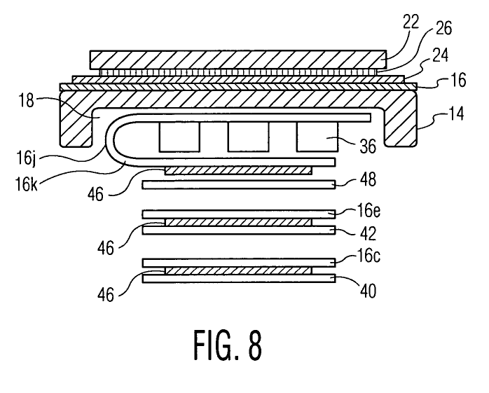

FIGS. 7 and 8 show another embodiment of a transducer in accordance with the invention. In this embodiment, another flexible circuit 48 is provided having appropriate connections for coax wires 38 such as connection sites or connector pads. The flexible circuit 48 is connected to a portion of the coax wires 38 by bonding exposed, conductive portions of the coax wires 38 to the connection sites of the flexible circuit 48, e.g., using a known bonding process. The flexible circuit 48 may be entirely flexible or have a flexible portion and a rigid portion, and might even be entirely rigid. Connection of the coax wires 38 to the flexible circuit 48 may be performed separate from the manufacture of the flexible circuit 48 with the acoustic assembly 20 and optional electronic components 36. By having three flexible circuits 40,42,48, the number of coax wires 38 on each flexible circuit 40,42,48 is less than the number when only two flexible circuits 40,42 are provided (assuming the same total number of coax wires 38) thereby further reducing the thickness of the transducer 10.

The flexible circuit 48 is connected to the flexible circuit 16 using an electrical interconnection such as z-axis conductive film or conductive adhesive 46. More specifically, the flexible circuit 48 is connected to a lateral flap portion 16k of the flexible circuit 16 which is separated from one lateral edge of the second planar portion 16b of the flexible circuit by a 180° bend 16j. To further reduce the thickness of the transducer 10, it is possible to provide another flap extending from the other lateral edge of the second planar portion 16b of the flexible circuit 16. It is also conceivable that flexible circuits may be used extending only from the lateral edges of one or both of the planar portions of the flexible circuit 16.

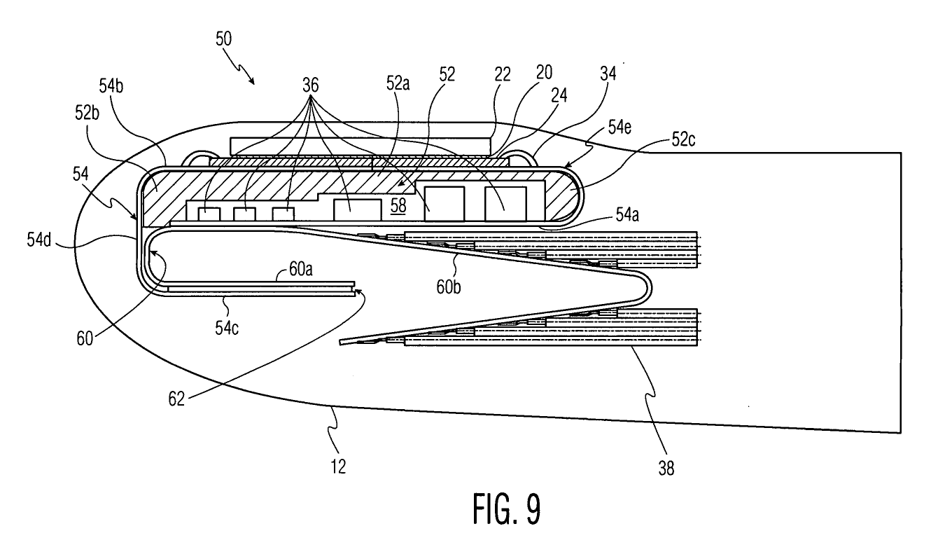

FIG. 9 shows another embodiment of a transducer in accordance with the invention. In this embodiment, the transducer 50 includes a thermally-conductive body 52 and a flexible circuit 54 which is bent around the body 52. By providing the flexible circuit 54 and coupling the components necessary for operation of the transducer 50 to the flexible circuit 54, the flexible circuit 54 can be bent into a desired shape to enable it to fit within the tip 12 of the examination device.

The body 52 has a central support portion 52a and leg portions 52b,52c, one at each end of the support portion 52a, with the flexible circuit 54 being supported by the support portion 52a and bent over the leg portions 52b, 52c. A cavity 58 is formed in the underside of the body 52 below the support portion 52a.

The flexible circuit 54 has a first terminal planar portion 54a facing the cavity 58, a second planar portion 54b above the support portion 52a of the body 52, a terminal end 54c separated from the second planar portion 54b by a one-hundred-eighty degree (180°) bend 54d and a one-hundred-eighty degree (180°) bend 54e separating the first terminal planar portion 54a from the second planar portion 54b. The terminal end 54c is substantially planar and situated below the body 52. The one-hundred-eighty degree (180°) bends 54d and 54e may include a pair of ninety degree (90°) bends separated by a straight portion as shown in FIG. 9 or be entirely arcuate. The form of the bends would depend in part on the shape of the body 52.

The flexible circuit 54 is not required to be flexible over its entire length to achieve the objects of the invention, but rather, at least those portions which are bent should be flexible. Other portions of the flexible circuit 54 which are not bent, such as those planar portions which support components of the transducer 50 described below, may be rigid. An acoustic assembly 20 is mounted to an upper surface of the second planar portion 54b of the flexible circuit 54 and in the preferred embodiment shown, includes an array of acoustic elements 22 and an integrated circuit 24. The mounting of the acoustic assembly 20 to the flexible circuit 54 may be the same as the mounting of the acoustic assembly 20 to the flexible circuit 16 described above, i.e., via wire bonds 34 connecting connection sites 30 on the flexible circuit 54 to connection sites 32 on the integrated circuit 24. The flexible circuit 54 may have an opening to enable the integrated circuit 24 to be in direct contact with the body 52.

Electronic components 36 required for operation and control of the transducer 50 are mounted to the first planar portion 54a such that the components 36 are located in the cavity 58. The cavity 58 is thus formed with a shape designed to accommodate the electronic components 36. It should be noted that in view of the bending of the flexible circuit 54 around the body 52, the acoustic assembly 20 and components 36 are mounted on opposite sides of the flexible circuit 54 during the manufacture of the transducer 50 (described below).

To connect the flexible circuit 54 to a plurality of coax wires 38 leading from the examination device to associated equipment, such as a monitor and recording device, an additional flexible circuit 60 is used and has appropriate connections for coax wires 38 such as connection sites or connector pads. The flexible circuit 60 has a U-shaped portion 60a, with one leg opposite the terminal end 54c of the flexile circuit 54 and the other leg opposite the first planar portion 54a of the flexible circuit 54, and a V-shaped portion 60b having two planar sections. The planar sections of the V-shaped portion 60 are connected to the coax wires 38 by bonding exposed, conductive portions 38a of the coax wires 38 to the connector sites of the flexible circuit 60 using a known bonding process. The flexible circuits 60 may be entirely flexible or have a flexible portion or portions and a rigid portion or portions.

The flexible circuit 60 is connected to the flexible circuit 54 (the terminal end 54c of the flexible circuit 54 being connected to the opposed leg of the U-shaped portion 60a of the flexible circuit 60) using an electrical interconnection such as z-axis conductive film or conductive adhesive 62. In this manner, an electrical connection between the flexible circuit 54 and the coax wires 38 is provided via the flexible circuit 60 and the adhesive 62. Instead of z-axis conductive film or adhesive, it is possible to use a z-axis conductive elastomeric connector or reflow solder.

Instead of mounting the electronic components 36 to the flexible circuit 54, electronic components or electronics for controlling the acoustic assembly 20 may be mounted on the flexible circuit 60 or at the end of the coax wires 38 distanced from the transducer 10. The electronic components could also be integrated into the integrated circuit 24.

To manufacture the transducer 50, the body 52 is formed and the flexible circuit 54 is formed and cut to the necessary size to enable it to be bent around the body 52. The acoustic assembly 20 and the electronic components 36 are mounted to opposite sides of the flexible circuit 54 in connection with or after the formation of the flexible circuit 54. To enable mounting of the acoustic assembly 20 to the flexible circuit 54, adhesive is applied to the underside of the integrated circuit 24. The mounting locations of the acoustic assembly 20 and electronic components 36 are selected to position the acoustic assembly 20 above the cavity 58 and the electronic components 36 in the cavity 58 as shown in FIG. 9. The connection sites on the acoustic assembly 20 are connected to the connection sites on the flexible circuit 54 using wire bonds 34. The acoustic assembly 20 may be pre-formed by mounting the stack of acoustic elements 22 on the integrated circuit 24 and connecting them using a flip-chip interconnection technique.

Flexible circuit 60 is formed with the required connector sites for electrical connection with the flexible circuit 54 and the coax wires 38 and then attached to the coax wires 38. The flexible circuit 60 is also attached to the terminal end 54c of the flexible circuit 54 using z-axis conductive film or conductive adhesive 62. The flexible circuit 70 may be attached to the coax wires 38 first and then to the flexible circuit 54 or vice versa. Once the acoustic assembly 20, electronic components 36 and flexible circuit 60 (preferably with the coax wires 38 attached thereto) are attached to the flexible circuit 54, adhesive is applied to the portions of the flexible circuit 54 which will come into contact with the body 52 (and/or applied to portions of the body 52 against which the flexible circuit 54 will rest) and the flexible circuit 54 is bent around the body 52 such that the planar portion 54b of the flexible circuit 54 having the acoustic assembly 20 mounted thereon is situated on the support portion 52a of the body 52, the planar portion 54a having the electronic components 36 mounted thereon is situated below the cavity 58 in the body 52 with the electronic components 36 being situated in the cavity 58, and the terminal portion 54c having the flexible circuit 60 attached thereto is situated underneath the body 52. Further, bending of the flexible circuit 54 over the body 52 is performed such that the bend 54d of the flexible circuit 54 is situated partially over the leg portion 52b of the body 52 and the bend 54e is situated over the leg portion 52c of the body 52. The acoustic assembly 20, the electronic components 36 and the attachment mechanism for attaching the flexible circuit 54 to the coax wires 38 are thus all positioned in a vertical arrangement thereby reducing the horizontal size of the transducer. A compact transducer is thus provided which can fit in the tip of a transesophageal examination device (line 12 as shown in FIG. 9).

The embodiments shown in the drawings use coax wires 38. However, the invention also contemplates the use of other types of signal transmission lines, including but not limited to, flat ribbon cables and long flexible circuits. Signal transmission lines for use in the invention would include a electrically-conducting element which would be electrically coupled to the connector sites on the flexible circuits.

Although illustrative embodiments of the present invention have been described herein with reference to the accompanying drawings, it is to be understood that the invention is not limited to these precise embodiments, and that various other changes and modifications may be effected therein by one of ordinary skill in the art without departing from the scope or spirit of the invention.

1. An ultrasonic transducer, comprising:
a housing;
acoustic elements arranged in said housing;
an integrated circuit arranged in said housing adjacent said acoustic elements;
first connection means for connecting said acoustic elements to said integrated circuit; and
second connection means for connecting said integrated circuit to electrical transmission lines, connection sites for said first and second connection means being arranged on a common surface of said integrated circuit.

2. The ultrasonic transducer of claim 1, wherein each of said first and second connection means are comprised of at least one of metal bumps, solder bumps, polymer bumps, thin-line bonding, z-axis conductive elastomeric connectors, z-axis conductive adhesive, z-axis conductive film and reflow solder.

3. The ultrasonic transducer of claim 1, wherein said first connection means are comprised of at least one of metal bumps, solder bumps, polymer bumps, thin-line bonding, z-axis conductive elastomeric connectors, z-axis conductive adhesive, z-axis conductive film and reflow solder, and said second connection means are different from said first connection means and are comprised of at least one of wire-bonds, direct wire attachments and tab bonding of leads.

4. The ultrasonic transducer of claim 1, wherein said second connection means comprise an intermediate interconnection substrate comprising a thin film circuit, ceramic circuit, laminate circuit technology.

5. The ultrasonic transducer of claim 1, wherein said second connection means comprise an intermediate interconnection substrate comprising a flexible circuit, a semi-rigid circuit or a rigid circuit.

6. The ultrasonic transducer of claim 5, wherein said interconnection substrate is bent such that a vertical size of an assembly of said acoustic elements, said integrated circuit and said interconnection substrate is less than seventy-five percent of a horizontal length of said integrated circuit.

7. The ultrasonic transducer of claim 5, wherein said interconnection substrate is bent such that a vertical size of an assembly of said acoustic elements, said integrated circuit and said interconnection substrate is less than fifty percent of a horizontal length of said integrated circuit.

8. An ultrasonic transducer, comprising:
a thermally-conductive body;
a flexible circuit bent at least partially around said body;
an acoustic assembly connected to said flexible circuit;
electronic components for controlling said acoustic assembly; and
connection means for connecting signal transmission lines to said flexible circuit, said acoustic assembly, said electronic components and the signal transmission lines being connected in a circuit defined in part by said flexible circuit.

9. The ultrasonic transducer of claim 8, wherein said flexible circuit is bent around said body such that a first portion of said flexible circuit is on a first side of said body and a second portion of said flexible circuit is on a second side of said body opposite said first side of said body.

10. The ultrasonic transducer of claim 9, wherein said acoustic assembly is arranged on said first portion of said flexible circuit and said electronic components are arranged on said second portion of said flexible circuit.

11. The ultrasonic transducer of claim 8, wherein said acoustic assembly is arranged in contact with said body.

12. The ultrasonic transducer of claim 8, wherein said body defines a cavity, said electronic components being arranged on said flexible circuit and in said cavity.

13. The ultrasonic transducer of claim 8, wherein said flexible circuit has a 180° bend around said body such that a first portion of said flexible circuit is arranged on a first side of said body and a second portion of said flexible circuit is arranged on a second side of said body opposite said first side of said body.

14. The ultrasonic transducer of claim 8, wherein said acoustic assembly includes acoustic elements and an integrated circuit electrically coupled to said acoustic elements, said flexible circuit having connection sites and said integrated circuit having connection sites, further comprising wire-bonds connecting said connection sites of said integrated circuit and said connection sites of said flexible circuit.

15. The ultrasonic transducer of claim 14, wherein two rows of said wire-bonds are formed along each of a pair of opposed edges of said integrated circuit.

16. The ultrasonic transducer of claim 8, wherein said flexible circuit has a plurality of bends about said body.

17. The ultrasonic transducer of claim 8, wherein said flexible circuit has first and second planar portions on opposite sides of said body separated by a 180° bend and first and second terminal end portions each separated from a respective one of said first and second planar portions by a 180° bend.

18. The ultrasonic transducer of claim 17, wherein said connection means comprise two additional flexible circuits, each having connections for signal transmission lines, and conductive film adhesive attaching each of said additional flexible circuits to a respective one of said first and second terminal end portions of said flexible circuit.

19. The ultrasonic transducer of claim 18, wherein said flexible circuit has a flap portion separated from said first planar portion of said flexible circuit by a 180° bend, said connection means further comprise one additional flexible circuit having connections for signal transmission lines and conductive film or adhesive attaching said additional flexible circuit to said flap portion of said flexible circuit.

20. The ultrasonic transducer of claim 8, wherein said flexible circuit has a planar portion on one side of said body and a flap portion separated from said planar portion by a 180° bend, said connection means further comprise an additional flexible circuit having connections for signal transmission lines and conductive film or adhesive attaching said additional flexible circuit to said flap portion of said flexible circuit.

21. The ultrasonic transducer of claim 8, wherein said flexible circuit has first and second planar portions on opposite sides of said body separated by a 180° bend and a first terminal end portion separated from said first planar portion by a 180° bend, said second planar portion of said flexible circuit being a terminal portion of said flexible circuit.

22. The ultrasonic transducer of claim 21, wherein said connection means comprise an additional flexible circuit having connections for the signal transmission lines, and conductive film adhesive attaching said additional flexible circuit to said flexible circuit.

23. An ultrasonic transducer, comprising:
a flexible circuit having connection sites;
an acoustic assembly mounted on said flexible circuit and comprising an integrated circuit having connection sites and acoustic elements electrically coupled to said integrated circuit;
electronic components for controlling said acoustic assembly to transmit and receive ultrasonic waves, said acoustic assembly and said electronic components being connected in a circuit defined in part by said flexible circuit; and
wire-bonds connecting said connection sites of said integrated circuit and said connection sites of said flexible circuit.

24. The ultrasonic transducer of claim 23, wherein said wire-bonds are formed along only a portion of the periphery of said integrated circuit.

25. The ultrasonic transducer of claim 23, wherein two rows of said wire-bonds are formed along each of a pair of opposed edges of said integrated circuit.

26. A method for manufacturing an ultrasonic transducer, comprising the steps of: arranging an acoustic assembly on a flexible circuit;
coupling electronic components for controlling the acoustic assembly to the acoustic assembly via the flexible circuit;
coupling signal transmission lines to the flexible circuit such that the electronic components, the acoustic assembly and the signal transmission lines are connected in a circuit defined in part by the flexible circuit; and
bending the flexible circuit at least partially around a thermally-conductive body to form at least one 180° bend about the body with the acoustic assembly being vertically spaced from the electronic components.

27. The method of claim 26, wherein the acoustic assembly and electronic components are arranged on the flexible circuit when the flexible circuit is in a flat form and the body has cavity, the flexible circuit being bent to place the electronic components in the cavity.
